(19) United States
(12) Patent Application Publication
(10) Pub. No.: US 2018/0099157 A1
(43) Pub. Date: Apr. 12, 2018

(54) APPARATUS AND METHOD FOR LOCALIZING THE BRAGG PEAK OF A HADRON BEAM TRAVERSING A TARGET TISSUE BY MAGNETIC RESONANCE IMAGING

(71) Applicant: Ion Beam Applications S.A., Louvain-la-Neuve (BE)

(72) Inventors: Damien PRIEELS, Court-Saint-Etienne (BE); Erik VAN DER KRAAIJ, Rixensart (BE); Sébastien HENROTIN, Watermael-Boitsfort (BE); Caterina BRUSASCO, Bossière (BE)

(73) Assignee: Ion Beam Applications S.A.

(21) Appl. No.: 15/727,379

(22) Filed: Oct. 6, 2017

(30) Foreign Application Priority Data
Oct. 7, 2016 (EP) 16192739.7

(51) Int. Cl. A61N 5/10; A61B 5/055; A61B 5/00

(52) U.S. Cl. CPC A61N 5/1048; A61B 5/055; A61N 5/103; A61B 5/742; A61N 5/1077

(57) ABSTRACT

The present disclosure relates to a method and a medical apparatus for visualizing a hadron beam traversing an organic body. In one implementation, the method may include capturing a magnetic resonance (MR) image including a volume of irradiated excitable atoms surrounding a hadron beam and having a magnetic susceptibility modified by the hadron beam captured as a hyposignal. For example, a hyposignal may be obtained by saturating the spins of the irradiated excitable atoms before capturing an MR image based on excitation of excitable atoms not affected by the hadron beam.

### CROSS-REFERENCE TO RELATED APPLICATIONS

This application claims the benefit of priority to European Application No. 16192739.7, filed Oct. 7, 2016, the contents of which are incorporated herein by reference.

### TECHNICAL FIELD

The present disclosure relates to a medical apparatus comprising a charged hadron therapy device coupled to a magnetic resonance imaging device (MRI) adapted for visualizing in situ the position of the Bragg peak of a hadron beam traversing a target tissue relative to the position of a target spot in said target tissue. The in situ localization of the actual position of the Bragg peak relative to the target spot, immediately before a hadron therapy session starts, may be highly useful for validating the planned position of the Bragg peak of the hadron beam determined during an earlier established treatment plan for treating said target spot. If a discrepancy appears between the planned and actual positions of the Bragg peak of the hadron beam, embodiments of the present disclosure may allow the correction of the initial energy, E1, of the hadron beam required for positioning the Bragg peak over the target spot. Accordingly, the hadron therapy session may not need to be cancelled and instead proceed with corrected parameters.

### BACKGROUND

Hadron therapy (for example, proton therapy) for treating a patient may provide several advantages over conventional radiotherapy. These advantages are generally due to the physical nature of hadrons. For example, a photon beam in conventional radiotherapy releases its energy according to a decreasing exponential curve as a function of the distance of tissue traversed by the photon beam. By contrast, and as illustrated in the example of FIG. 2A, a hadron beam first releases a small fraction of its energy as it penetrates tissues 41-43, forming a plateau, then, as the hadron path is prolonged, releases energy locally following a steep increase to a peak and a fall-off at the end of the range of the beam. The peak is called a Bragg peak and corresponds to the maximum of the Bragg curve illustrated in the example of FIG. 2C. Consequently, a hadron beam may deliver a high dose of hadrons at a precise location within a target tissue 40 and may therefore preserve the surrounding healthy tissues 41-44. As illustrated in the example of FIG. 2A, if the position, BP0, of the Bragg peak of a hadron beam is offset relative to the target tissues 40, high doses of hadrons may be delivered to adjacent tissues 43, 44, which are healthy (as illustrated with solid line, E0, and dashed line, E0d, of the curves of energy loss, Eₗₒₛₛ, with respect to the distance, Xh, travelled by the hadron beam within tissues and measured along the beam path, Xp, in the example of FIG. 2A). For this reason, the determination of the relative position of the Bragg peak with respect to the position of the target tissue is often crucial to properly implement hadron therapy to a patient.

In practice, hadron therapy usually requires the establishment of a treatment plan before any treatment can start. During this treatment plan, a computer tomography scan (CT scan) of the patient and target tissues is generally performed. The CT scan may be used to characterize the target tissue 40 and the surrounding tissues 41-43 to be traversed by a treatment hadron beam 1h for the treatment of a patient. The characterization may yield a 3D representation of the volume comprising the target tissue, and a treatment plan system may determine a range-dose calculated based on the nature of the tissues 41-43 traversed by the hadron beam.

This characterization may allow computation of a water equivalent path length (WEPL), which may be used for determining the initial energy, Ek, of the treatment hadron beam required for delivering a prescribed dose of hadrons to a target spot 40s, wherein k=0 or 1, depending on the stage when said initial energy was determined. The example of FIG. 2C illustrates the conversion of the physical distances travelled by a hadron beam traversing different tissues into corresponding WEPLs. The WEPL of a hadron beam travelling a given distance through a given tissue is the equivalent distance said hadron beam would travel in water. As illustrated in the example of FIG. 2C if, as is often the case, healthy tissues 41-43 of different natures and thicknesses separate a target tissue from the outer surface of the skin of a patient, the WEPL of a target spot may be calculated taking into account the water corresponding path lengths of each tissue in series until the target spot is reached. With a value of the equivalent path length of a hadron beam traveling in water, the initial energy, Ek, required for positioning the Bragg peak at the WEPL of the target spot may be computed and correspond to the initial energy, Ek, required for positioning the Bragg peak at the target spot within the target tissue.

The treatment plan may then be executed during a treatment phase including one or more treatment sessions during which doses of hadrons are deposited onto the target tissue. The position of the Bragg peak of a hadron beam with respect to the target spots of a target tissue, however, may suffer of a number of uncertainties including, for example:
the variations of the patient position, on the one hand, during a hadron therapy session and, on the other hand, between the establishment of the treatment plan and the hadron therapy session;
the variations of the size and/or of the position of the target tissue (see, for example, FIG. 2B) and/or of the healthy tissues 41-43 positioned upstream from the target tissue with respect to the hadron beam; and
the range calculation from CT scans being limited by the quality of the CT images. Another limitation is linked to the fact that CT scans use the attenuation of X-rays that have to be converted in hadron attenuation, which may depend on the chemical composition of the tissues traversed.

The uncertainty on the position of the patient and, in particular, of the target tissue may be critical. Even with an accurate characterization by CT scan, the actual position of a target tissue during a treatment session may remain difficult to ascertain for the following reasons:
(A) first, during an irradiation session, the position of a target tissue may change because of anatomical processes such as breathing, digestion, or heartbeats of the patient. Anatomical processes may also cause gases or fluids appearing or disappearing from the beam path, Xp, of a hadron beam.
(B) second, treatment plans are generally determined several days or weeks before a hadron treatment session starts and treatment of a patient may take several weeks distributed over several treatment sessions. During this time period, the patient may lose or gain weight, therefore modifying, sometimes significantly, the volume of tissues such as fats and muscles.

Accordingly, the size of the target tissue may change (e.g., a tumour may have grown, receded, or changed position or geometry). The example of FIG. 2B shows an example of evolution of the size and position of a target tissue 40 between the time, t0, of the establishment of the treatment plan and the times, t0+Δt1, t0+Δt2, t1=t0+Δt3, of treatment sessions. The treatment plan and last treatment session may be separated by several days or weeks. The treatment plan established at time, to, may therefore comprise irradiation of a target spot 40si,j (black spot in the example of FIG. 2B), which belonged to the target tissue 40p at said time, t0. Because the target tissue 40p may have moved or changed shape during the time period, Δt3, said target spot 40si,j may not belong to the target tissue 40 anymore at the time, t0+Δt3, of the treatment session and may be located in a healthy tissue instead. Consequently, irradiating said target spot may hit and possibly harm healthy tissues 43 instead of target tissues 40.

The use of a magnetic resonance imaging device (MRI) coupled to a hadron therapy device has been proposed for identifying any variation of the size and/or the position of a target tissue. For example, U.S. Pat. No. 8,427,148 generally relates to a system comprising a hadron therapy device coupled to an MRI. Said system may acquire images of the patient during a hadron therapy session and may compare these images with CT scan images of the treatment plan. FIG. 1 illustrates an example of a flowchart of a hadron therapy session using a hadron therapy device coupled to an MRI. A treatment plan may be established including the characterization of the target tissue 40s and surrounding tissues 41-43. This step is generally performed with a CT scan analysis and allows the determination of the position, P0, and morphology of a target tissue, the best trajectories or beam paths, Xp, of hadron beams for the hadron treatment of the target tissue, and characterization of the sizes and natures of the tissues traversed by a hadron beam following said beam paths, Xp, to determine WEPLs of target spots of the said target tissue. The initial energies, Ek, of the hadron beams required for matching the corresponding positions, BP0, of the Bragg peaks of the hadron beams to the position, P0, of the target tissue may thus be calculated. This generally completes the establishment of a treatment plan.

A hadron therapy session may follow the establishment of the treatment plan. With an MRI coupled to a hadron therapy device, it may be possible to capture a magnetic resonance (MR) image of a volume, Vp, including the target tissue and surrounding tissues to be traversed by a hadron beam. The MR image may then be compared with CT scan images to assess whether any morphological differences, Δ, can be detected in the imaged tissues between the time the CT scans were performed (=t0 in the example of FIG. 2B) and the time of the hadron therapy session (t1=t0+Δt3 in the example of FIG. 2B). If no substantial difference in morphology affecting the treatment session is detected, then the hadron therapy session may proceed as planned in the treatment plan. If, on the other hand, some differences are detected that could influence the relative position of the target tissue with respect to the planned hadron beams and their respective Bragg peaks, the hadron therapy session may be interrupted and a new treatment plan established. This technique may prevent carrying out a hadron therapy session based on a treatment plan that has become obsolete, which may prevent healthy tissues from being irradiated instead of the target tissue.

The magnetic resonance (MR) images generally provide high contrast of soft tissue traversed by a hadron beam but, at the time of filing, have usually not been suitable for visualizing the hadron beam itself, let alone the position of the Bragg peak because:
MRI measures the density of hydrogen atoms in tissues but, at the time of filing, does not usually yield any identifiable information on the hadron stopping power ratio. The conversion from density of hydrogen atoms to the hadron stopping power ratio suffers from uncertainties similar to and yet generally less understood than those of the conversion from X-rays in CT scan.
Due to the different techniques used in CT scan and in MRI, the comparison between the images from CT scan and the images from MRI may suffer from uncertainties.

In conclusion, in hadron therapy, an accurate determination of the position of the Bragg peak relative to the portion of a target tissue is important because errors regarding this position may lead to the irradiation of healthy tissues rather than irradiation of target tissues. However, no satisfactory solution for determining the relative positions of the Bragg peak and target tissues is presently available. Apparatuses combining a hadron therapy device and an MRI may allow in situ acquisition of images during a treatment session, thus giving information related to the actual position of the target tissue. images generated by the foregoing systems are, however, generally insufficient for ensuring a precise determination of the position of the Bragg peak of a hadron beam and of its location relative to the target tissue. Accordingly, there remains a need for a hadron therapy device combined with an MRI that allows a better determination of the position of the Bragg peak relative to the position of a target tissue.

### SUMMARY

In one embodiment according to the present disclosure, a method for visualizing a hadron beam traversing an organic body may comprise:
(a) determining:
   the Larmor rest frequency, fLm,0, of an excitable atom present in a target tissue m of a subject of interest, wherein m=40, corresponding to a target tissue, and exposed to a uniform magnetic field, B0, and
   the Larmor irradiated frequency, fLm,1, of irradiated excitable atoms defined as the excitable atoms present in the same tissue m, and exposed to the effects of a hadron beam of initial energy, Ek, with k=0 or 1, traversing said target tissue positioned in the same magnetic field, B0, and
(b) calculating the frequency shift, ΔfLm=|fLm,1−fLm,0|, in the target tissue, with m=40 as depicted in the examples of of FIGS. 2A and 2B;
(a) providing a magnetic resonance imaging device (MRI) for acquiring magnetic resonance data within an imaging volume, Vp, including the target tissue, positioned in the uniform main magnetic field, B0;
(c) providing a hadron source adapted for directing a hadron beam having an initial energy, E0, along a beam path, Xp, intersecting the target tissue in the imaging volume;
(d) acquiring magnetic resonance data from the imaging volume, by at least an excitation step (MRe), the excitation step comprising:
   an A1-saturation step, comprising creating n bursts of a saturating electromagnetic field, B1-sat, wherein n is an integer greater than 0, which oscillates at a frequency range, [fL1], of band width, b1<2·ΔfL40, and centred on the Larmor irradiated frequency, fL40,1, and excluding the Larmor rest frequency, fL40,0, such as to bring the nuclei of the irradiated excitable atoms to a saturated state wherein a net polarization vector of the spins may be reversed at an angle between 100 and 180° with respect to the net polarization vector of the spins of said nuclei at rest (i.e., absent B1-sat) and, after a time, Δts-e, following the n^{th} burst B1-sat:
   an A0-excitement step comprising creating p bursts of an exciting electromagnetic field, B1-exc, oscillating at a frequency range, [fL0], centred on the Larmor rest frequency, fL40,0, wherein p is an integer greater than 0, such as to bring to an excited state the excitable atoms which are not affected by the hadron beam and which were therefore not brought to a saturated state by the saturating electromagnetic field, B1-sat,
(b) directing a hadron beam having the initial energy, E0, along a beam path intersecting said target body in a number, N, of hadron pulses of pulse periods, PBi, wherein, N is an integer greater than 0;
(c) representing on a display the organic body from the magnetic resonance data acquired by the MRI within the imaging volume, Vp, and
(d) on the display, visualizing the beam path in the target tissue as a hyposignal, weaker than the signal generated by the excitable atoms (A0) that are not exposed significantly to the effects of the hadron beam.

In some embodiments, the N hadron pulses may overlap with at least 50% of the n bursts of the saturating electromagnetic field, B1-sat during the A1-saturation step. For example, the N hadron pulses may overlap with at least 70%, e.g., at least 80%, at least 90%, or 100% of the n bursts of the saturating electromagnetic field, B1-sat. The N hadron pulses may be in phase with the n bursts of the saturating electromagnetic field, B1-sat.

The N hadron pulses may have a period, PBi, between 10 μs and 30 ms. Depending on the type of hadron source, the period PBi may be between 1 md and 10 ms or, alternatively, between 5 ms and 20 ms. The time interval, ΔPBi, between two consecutive hadron pulses may be between 1 and 20 ms. A short interval between hadron pulses may reduce the treatment time.

The period of each of the n bursts of the saturating electromagnetic field, B1-sat, may be between 1 and 20 ms, e.g., between 2 and 10 ms. The time period, Δts-e, separating the last burst of the n saturating electromagnetic field, B1-sat, and the first burst of the p exciting electromagnetic field, B1-exc, may either be:
not more than 50% of a longitudinal relaxation time, T1(A0), of the excitable atoms (A0) not affected substantially by the hadron beam, wherein Δts-e may be not more than 100 ms/T; or
within ±20% of the time, tM0, required for the longitudinal component, Mz, parallel to B0 of the net polarization vector of the irradiated excitable atoms to pass from the saturated state to a value of zero, and wherein Δts-e may be not more than 50 ms/T.

The n bursts of saturating electromagnetic field, B1-sat may be adiabatic bursts.

The target tissue may be a tumour exposed to a uniform magnetic field, B0, and traversed by a hadron beam of initial energy, E0. The frequency shift, ΔfLm, at the level of the Bragg peak of said hadron beam may be between 60 and 6000 Hz, e.g., between 200 and 1200 Hz in a main magnetic field, e.g., B0=1.5 T. In this example, the relative frequency shift, ΔfLmr=ΔfLm/flm0, may thus range from 0.9 ppm to 93 ppm, e.g., from 3 ppm to 16 ppm.

The imaging volume, Vp, may be controlled by creating a magnetic gradient along, one, two, or three of the first, second, and third directions, X1, X2, X3. Accordingly, a thickness of the imaging volume along said first, second, or third directions, X1, X2, X3 may be controlled.

A treatment session may be planned in two steps: first at time, t0, leading to the establishment of a treatment plan, and second at a time, t1>t0, when a therapy session is to take place, and during which it may be assessed whether the validity of the results established in the treatment plan are still applicable at time, t1. In particular, the method may comprise:
(a) establishing at day, t0, a treatment plan and determining an initial energy, E0, of a hadron beam for depositing a given dose of hadrons to a target spot,
(b) comparing on the display the morphology and thicknesses of the tissues traversed by a hadron beam of initial energy, E0, at day, t1>t0, with the morphology and thicknesses of the same tissues as defined in the treatment plan, at day, t0,
(c) visualizing on the same display the actual position of the Bragg peak of the hadron beam, and
(d) in case of mismatch between the actual position of the Bragg peak and of the target tissue, correcting the initial energy, E1, of the hadron beam required for the Bragg peak to fall over the target spot.

Embodiments of the present disclosure also include a medical apparatus, which may comprise:
(a) a hadron source adapted for irradiating a target tissue (40) with a hadron beam (1h) having a beam energy, Ek, with k=0 or 1, along a beam path in a number, N, of hadron pulses;
(b) a magnetic resonance imaging device (MRI) for the acquisition of magnetic resonance (MR) images within an imaging volume, Vp, including the target tissue (40), as the target tissue is being irradiated,
(c) a controller configured for controlling the hadron source and for acquiring magnetic resonance images by implementing the following steps:
   creating a main magnetic field, B0, in the imaging volume, Vp, including the target tissue,
   creating n bursts of a saturating electromagnetic field, B1-sat, wherein n is an integer greater than 0, which oscillates at a frequency range, [fL1], of band width, b1<2·ΔfL40, and centred on the Larmor irradiated frequency, fL40,1, and excluding the Larmor rest frequency, fL40,0, wherein
      fL40,0 is the rest Larmor frequency of excitable atoms present in the target tissue;
      fL40,1 is the irradiated Larmor frequency of irradiated excitable atoms (A1) defined as the excitable atoms present in the same target tissue, and exposed to the effects of a hadron beam of initial energy, E0, traversing said target tissue positioned in the same magnetic field, B0, and wherein
      ΔfL40=|fL40,1−fL40,0|;
   after a time, Δts-e, following the nth burst B1-sat, creating m bursts of an exciting electromagnetic field, B1-exc, oscillating at a frequency range, [fL0], centred on the Larmor rest frequency, fL40,0, wherein m is an integer greater than 0; and
   directing a hadron beam having the initial energy, E0, along a beam path intersecting said target tissue in a number, N, of hadron pulses of pulse periods, PBi, wherein, N is an integer greater than 0; and
(d) a display for representing the target tissue from the magnetic resonance data acquired by the MRI within the imaging volume, Vp, and for visualizing the beam path in the target tissue as a hyposignal weaker than the signal generated by the excitable atoms which are not exposed significantly to the effects of the hadron beam,
   characterized in that, said controller may be further configured for synchronizing the N hadron pulses to overlap with at least 50% of the n bursts of the saturating electromagnetic field, B1-sat.

### BRIEF DESCRIPTION OF THE DRAWINGS

These and further aspects of the present disclosure will be explained in greater detail by way of example and with reference to the accompanying drawings in which:

FIG. 1 shows a flowchart of a hadron therapy method using a hadron therapy device coupled to a MRI.

FIG. 2A schematically shows the position of the Bragg peak of a hadron beam traversing tissues.

FIG. 2B schematically shows changes with time of the morphology and position of a target tissue that can create a discrepancy between a treatment plan and an actual required treatment.

FIG. 2C schematically shows the relationship between actual path lengths and water equivalent path lengths.

FIG. 3A schematically shows a medical apparatus comprising a hadron therapy device coupled to an MRI, according to an example embodiment of the present disclosure.

FIG. 3B schematically shows another medical apparatus comprising a hadron therapy device coupled to an MRI, according to another example embodiment of the present disclosure.

FIG. 4A schematically illustrates a nozzle mounted on a gantry for delivering a therapeutic dose of hadron, according to an example embodiment of the present disclosure.

FIG. 4B illustrates volumes of target tissue receiving a therapeutic dose of hadron from the nozzle of FIG. 4A, according to an example embodiment of the present disclosure.

FIG. 4C illustrates a dose of hadron delivered to the target tissue of FIG. 4B, according to an example embodiment of the present disclosure.

FIG. 5A schematically shows a selection of an imaging slice in an MRI, according to an example embodiment of the present disclosure.

FIG. 5B schematically shows a creation of phase gradients and frequency gradients during imaging of the slice of FIG. 5A, according to an example embodiment of the present disclosure.

FIG. 6A shows an example of an apparatus according to an example embodiment of the present disclosure, showing access of a hadron beam to a target tissue.

FIG. 6B shows another example of an apparatus according to another example embodiment of the present disclosure, showing access of a hadron beam to a target tissue.

FIG. 7 shows magnetic data acquisition steps (a), (c), and (d) for imaging a volume by MRI, juxtaposed with a relaxation process (b) of the spin of one excitable atom A0, according to an example embodiment of the present disclosure.

FIG. 8A shows the relaxation of an excited atom from a saturated state at 180° with the spins of the atoms out of phase, according to an example embodiment of the present disclosure.

FIG. 8B shows the relaxation of an excited atom from an excited state at 90° with the spin of the atoms in phase, according to an example embodiment of the present disclosure. M/M0 represents the relative magnetic moment, with M0 representing the maximum value of said magnetic moment, M.

FIG. 9A shows an example frequency shift, ΔfLm, between the irradiated and rest Larmor frequencies, fLm1 and fLm0, of an excitable atom irradiated or not by a hadron beam, according to an example embodiment of the present disclosure.

FIG. 9B shows an example sequence of the excitation step (b), MRe, for acquiring MR data juxtaposed with the pulses (c) of a hadron beam required for visualizing at least part of the beam path of the hadron beam, juxtaposed with the spins (d) of the excitable atoms A0 and A1 at the different stages of the excitation step, according to an example embodiment of the present disclosure.

FIG. 10A illustrates the frequency shift, ΔfL40, between the irradiated and rest Larmor frequencies, fL40,1 and fL40,0, of an example excitable atom in a target tissue irradiated or not by a hadron beam, according to an example embodiment of the present disclosure.

FIG. 10B illustrates a cut of a tissues traversed by a hadron beam from an upstream boundary to a target spot, with the localization of the irradiated excitable atoms A1 indicated with dashed lines, according to an example embodiment of the present disclosure.

FIG. 10C illustrates the corresponding Eₗₒₛₛ curve of the hadron beam of the example of FIG. 10B, according to an example embodiment of the present disclosure.

FIG. 10D illustrates a schematic representation of an example MRI image with the beam path of the hadron beam visible as a hyposignal, according to an example embodiment of the present disclosure.

FIG. 10E illustrates a schematic representation of another example MRI image with the beam path of the hadron beam visible as a hyposignal, according to another example embodiment of the present disclosure.

FIG. 11 shows a flowchart of a hadron therapy method according to an example embodiment of the present disclosure.

The figures are not drawn to scale.

### DETAILED DESCRIPTION

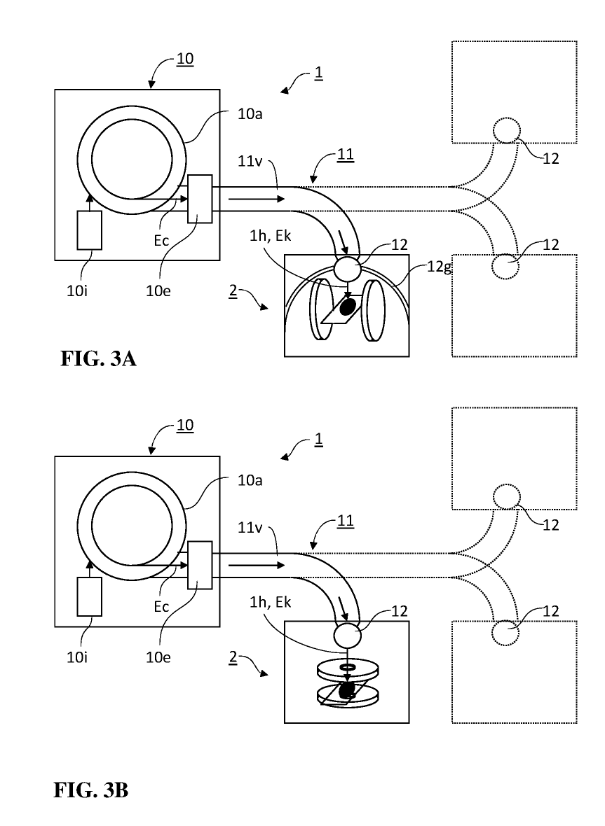

FIGS. 3A and 3B illustrate two examples of a medical apparatus comprising a hadron therapy device 1 coupled to a magnetic resonance imaging device (MRI) 2 according to embodiments of the present disclosure. A hadron therapy device, an MRI, and the combination of the two are described in greater details in the following description.

### Hadron Therapy Device

Hadron therapy is a form of external beam radiotherapy using beams 1h of energetic hadrons. FIGS. 3A, 3B, 4A, 6A, and 6B show a hadron beam 1h directed towards a target spot 40s in a target tissue 40 of a subject of interest. Target tissues 40 of a subject of interest typically include cancerous cells forming a tumour. During a hadron therapy session, a hadron beam of initial energy, Ek, with k=0 or 1, may irradiate one or more target spots within the target tissue, such as a tumour, and destroy the cancerous cells included in the irradiated target spots, reducing the size of the treated tumour by necrosis of the irradiated tissues.

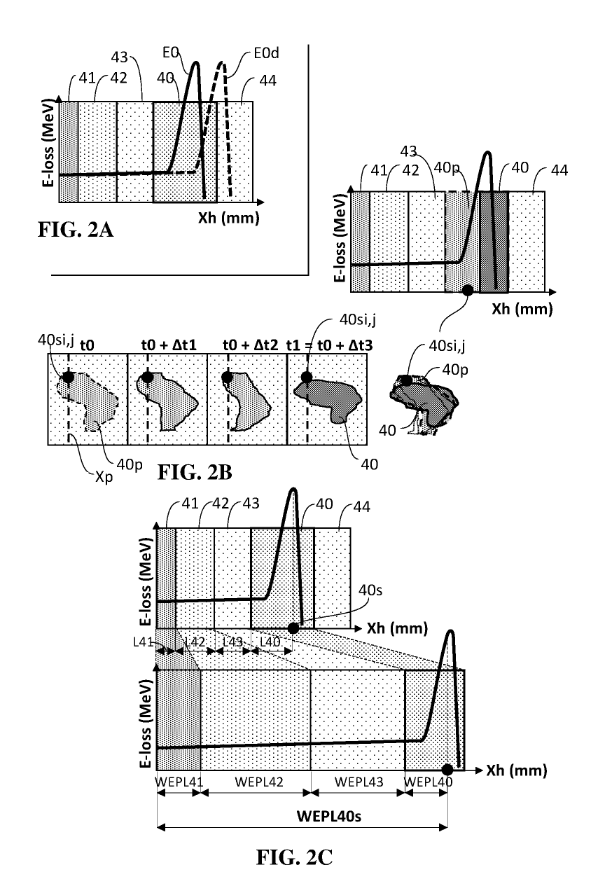

The subject of interest may comprise a plurality of materials including organic materials. For example, the subject of interest may comprise a plurality of tissues m, with m=40-44 as shown in the example of FIGS. 2A, 2B, and 2C, that may be, for example, skin, fat, muscle, bone, air, water (and/or blood), organ, tumour, or the like. For example, the target tissue 40 may be a tumour.

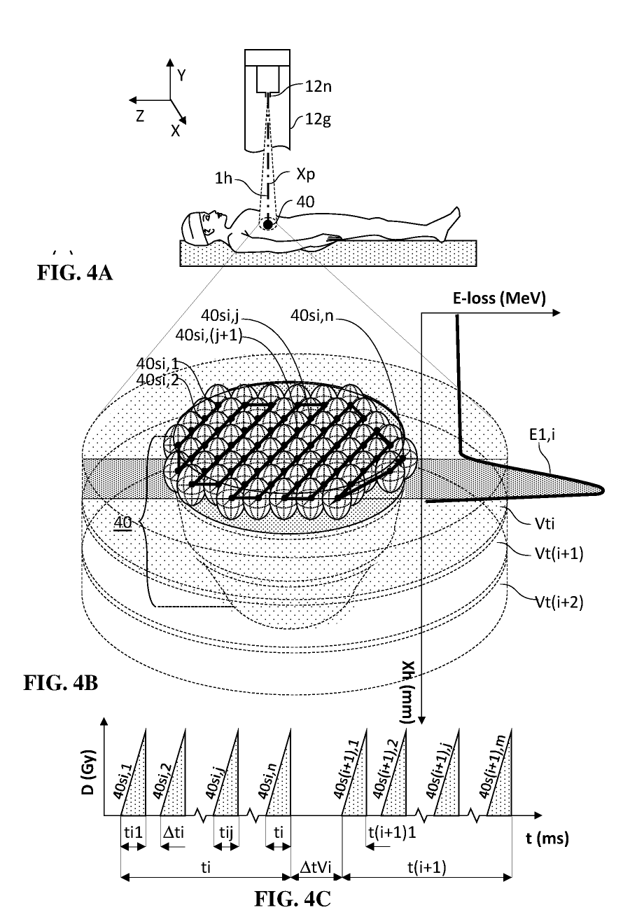

A hadron beam 1h traversing an organic body along a beam path, Xp, generally loses most of its energy at a specific distance of penetration along the beam path, Xp. As illustrated in FIGS. 2A, 2B, 2C, and 4B, said specific distance of penetration may correspond to the position of the Bragg peak, observed when plotting the energy loss per unit distance [MeVg⁻¹ cm⁻²], Eₗₒₛₛ, of a hadron beam as a function of the distance, xh, measured along the beam path, Xp. Unlike other forms of radiation therapies, a hadron beam may therefore deliver a high dose of energy at a very specific location within a target tissue corresponding to the position of the Bragg peak. The position of the Bragg peak may depend mainly on the initial energy, Ek, of the hadron beam (i.e., before traversing any tissue) and on the nature and thicknesses of the traversed tissues. The hadron dose delivered to a target spot may depend on the intensity of the hadron beam and on the time of exposure. The hadron dose may be measured in Grays (Gy), and the dose delivered during a treatment session is usually of the order of one to several Grays (Gy).

A hadron is a composite particle made of quarks held together by strong nuclear forces. Typical examples of hadrons may include protons, neutrons, pions, heavy ions, such as carbon ions, and the like. In hadron therapy, electrically charged hadrons are often used. For example, the hadron may be a proton, and the corresponding hadron therapy may be referred to as proton therapy. Accordingly, in the following description, unless otherwise indicated, any reference to a proton beam and/or proton therapy may apply to a hadron beam and/or hadron therapy in general.

A hadron therapy device 1 generally comprises a hadron source 10, a beam transport line 11, and a beam delivery system 12. Charged hadrons may be generated from an injection system 10i, and may be accelerated in a particle accelerator 10a to build up energy. Suitable accelerators may include, for example, a cyclotron, a (synchro-)cyclotron, a synchrotron, a laser accelerator, or the like. For example, a (synchro-)cyclotron may accelerate charged hadron particles from a central area of the (synchro-)cyclotron along an outward spiral path until the particles reach the desired output energy, Ec, whence they may be extracted from the (synchro-)cyclotron. Said output energy, Ec, reached by a hadron beam when extracted from the (synchro-)cyclotron is typically between 60 MeV and 400 MeV, e.g., between 210 MeV and 250 MeV. The output energy, Ec, may be, but is not necessarily, the initial energy, Ek, of the hadron beam used during a therapy session. For example, Ek may be equal to or lower than Ec, such that Ek≤Ec. An example of a suitable hadron therapy device may include, but is not limited to, a device described in U.S. Pat. No. 4,870,287, the entire disclosure of which is incorporated herein by reference as representative of a hadron beam therapy device used in the present disclosure.

The energy of a hadron beam extracted from a (synchro-)cyclotron may be decreased by energy selection means 10e, such as energy degraders or the like, positioned along the beam path, Xp, downstream of the (synchro-)cyclotron. Energy selection means 10e may decrease the output energy, Ec, down to any value of Ek, including down to nearly 0 MeV. As discussed supra, the position of the Bragg peak along a hadron beam path, Xp, traversing specific tissues may depend on the initial energy, Ek, of the hadron beam. By selecting the initial energy, Ek, of a hadron beam intersecting a target spot 40s located within a target tissue, the position of the Bragg peak may be controlled to correspond to the position of the target spot.

A hadron beam may also be used for characterizing properties of tissues. For example, images may be obtained with a hadron radiography system (HRS), for example, a proton radiography system (PRS). The doses of hadrons delivered to a target spot for characterization purposes, however, may be considerably lower than the doses delivered during a hadron therapy session, which, as discussed supra, may be of the order of 1 to 10 Gy. The doses of delivered hadrons of HRS for characterization purposes are typically of the order of 10⁻³ to 10⁻¹ Gy (i.e., one to four orders of magnitude lower than doses typically delivered for therapeutic treatments). These doses may have no significant therapeutic effects on a target spot. Alternatively or concurrently, a treatment hadron beam delivered to a small set of target spots in a target tissue may be used for characterization purposes. The total dose delivered for characterization purposes may be insufficient to treat a target tissue.

As illustrated in FIGS. 3A and 3B, downstream of the hadron source, a hadron beam of initial energy, Ek, may be directed to the beam delivery system 12 through a beam transport line 11. The beam transport line may comprise one or more vacuum ducts, 11v, and a plurality of magnets for controlling the direction of the hadron beam and/or for focusing the hadron beam. The beam transport line may also be adapted for distributing and/or selectively directing the hadron beam from a single hadron source 10 to a plurality of beam delivery systems for treating several patients in parallel.

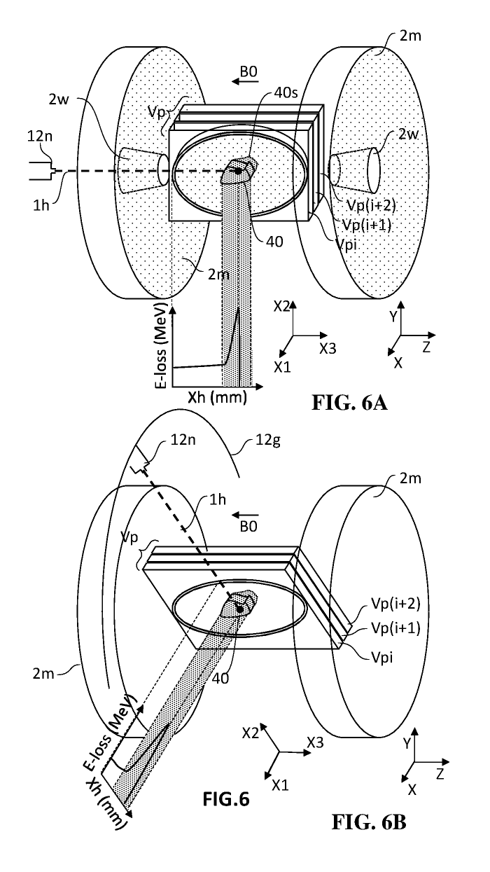

The beam delivery system 12 may further comprise a nozzle 12n for orienting a hadron beam 1h along a beam path, Xp. The nozzle may be fixed or mobile. Mobile nozzles are generally mounted on a gantry 12g, as illustrated schematically in the examples of FIGS. 4A and 6B. A gantry may be used for varying the orientation of the hadron outlet about a circle centred on an isocentre and normal to an axis, Z, which may be horizontal. In supine hadron treatment devices, the horizontal axis, Z, may be selected parallel to a patient lying on a couch (i.e., the head and feet of the patient are aligned along the horizontal axis, Z). The nozzle 12n and the isocentre define a path axis, Xn, whose angular orientation depends on the angular position of the nozzle in the gantry. By means of magnets positioned adjacent to the nozzle, the beam path, Xp, of a hadron beam 1h may be deviated with respect to the path axis, Xn, within a cone centred on the path axis and having the nozzle as apex (as depicted, for example, in FIG. 4A). Advantageously, this may allow a volume of target tissue centred on the isocentre to be treated by a hadron beam without changing the position of the nozzle within the gantry. The same applies to fixed nozzles with the difference that the angular position of the path axis may be fixed.

A target tissue to be treated by a hadron beam in a device provided with a gantry must generally be positioned near the isocentre. Accordingly, the couch or any other support for the patient may be moved; for example, it may typically be translated over a horizontal plane (X, Z), wherein X is a horizontal axis normal to the horizontal axis, Z, and translated over a vertical axis, Y, normal to X and Z, and may also be rotated about any of the axes X, Y, Z, so that a central area of the target tissue may be positioned at the isocentre.

To assist in the correct positioning of a patient with respect to the nozzle 12n according to a treatment plan previously established, the beam delivery system may comprise imaging means. For example, a conventional X-ray radiography system may be used to image an imaging volume, Vp, comprising the target tissue 40. The obtained images may be compared with corresponding images collected previously during the establishment of the treatment plan.

Depending on the pre-established treatment plan, a hadron treatment may comprise delivery of a hadron beam to a target tissue in various forms, including, for example, pencil beam, single scattering, double scattering, uniform scattering, and the like. Embodiments of the present disclosure may apply to all hadron therapy techniques. FIG. 4B illustrates schematically a pencil beam technique of delivery. As depicted in FIG. 4B, hadron beam of initial energy, Ek,1, may be directed to a first target spot 40s1,1, during a pre-established delivery time. The hadron beam may then be moved to a second target spot 40s1,2, during a pre-established delivery time. The process may be repeated on a sequence of target spots 40s1,j to scan a first iso-energy treatment volume, Vt1, following a pre-established scanning path. A second iso-energy treatment volume, Vt2, may be scanned spot-by-spot following a similar scanning path with a hadron beam of initial energy, Ek,2. As many iso-energy treatment volumes, Vti, as necessary to treat a given target tissue 40 may thus be irradiated following a similar scanning path. A scanning path may include several passages over a same scanning spot 40si,j. The iso-energy treatment volumes, Vti, may be volumes of target tissues which may be treated with a hadron beam of initial energy, Ek,i. The iso-energy treatment volumes, Vti, may be slice shaped, with a thickness corresponding approximately to the breadths of the Bragg peaks at the values of the initial energy, Ek,i, of the corresponding hadron beams, and with main surfaces of area only limited by the opening angle of the cone centred on the path axis, Xn, enclosing the beam paths, Xp, available for a given position of the nozzle in the gantry or in a fixed nozzle device. In embodiments with a homogeneous target tissue, the main surfaces may be substantially planar as illustrated in FIG. 4B. In embodiments where both target tissue 40 and upstream tissues 41-43 are not homogeneous in nature and thickness, the main surfaces of an iso-energy volume, Vti, may be bumpy. The egg-shaped volumes in FIG. 4B schematically illustrate the volumes of target tissue receiving a therapeutic dose of hadron by exposure of one target spot 40si,j to a beam of initial energy Ek,i.

The dose, D, delivered to a target tissue 40 is illustrated in FIG. 4C. As discussed supra, the dose delivered during a treatment session is usually of the order of one to several Grays (Gy). It may depend on the doses delivered to each target spot 40si,j, of each iso-energy treatment volume, Vti. The dose delivered to each target spot 40si,j may depend on the intensity, I, of the hadron beam and on the irradiation time tij on said target spot. The dose, Dij, delivered to a target spot 40si,j may therefore be the integral, Dij=∫I dt, over the irradiation time tij. A typical dose, Dij, delivered to a target spot 40si,j is generally of the order of 0.1-20 cGy. The dose, Di, delivered to an iso-energy treatment volume, Vti, may be the sum over the n target spots scanned in said iso-energy treatment volume of the doses, Dij, delivered to each target spot, Di=Σ Dij, for j=1 to n. The total dose, D, delivered to a target tissue 40 may thus be the sum over the p irradiated iso-energy treatment volumes, Vti, of the doses, Di, delivered to each energy treatment volume, D=Σ Di, for i=1 to p. The dose, D, of hadrons delivered to a target tissue may therefore be controlled over a broad range of values by controlling one or more of the intensity, I, of the hadron beam, the total irradiation time tij of each target spot 40si,j, and/or the number of irradiated target spots 40si,j. Once a patient is positioned such that the target tissue 40 to be treated is located at the approximate position of the isocentre, the duration of a hadron treatment session may depend on the values of:
the irradiation time, tij, of each target spot 40si,j,
the scanning time, Δti, for directing the hadron beam from a target spot 40si,j to an adjacent target spot 40si(j+1) of a same iso-energy treatment volume, Vti,
the number n of target spots 40si,j scanned in each iso-energy treatment volume, Vti,
the time, ΔtVi, required for passing from a last target spot 40si,n scanned in an iso-energy treatment volume, Vti, to a first target spot 40s(i+1),1 of the next iso-energy treatment volume, Vt(i+1), and/or
the number of iso-energy treatment volumes, Vti, in which a target tissue 40 may be enclosed.

The irradiation time, tij, of a target spot 40si,j is generally of the order of 1-20 ms. The scanning time, Δti, between successive target spots in a same iso-energy treatment volume may be very short, of the order of 1 ms. The time, ΔtVi, required for passing from one iso-energy treatment volume, Vti, to a subsequent iso-energy treatment volume, Vt(i+1), may be slightly longer because, for example, it may require changing the initial energy, Ek, of the hadron beam. The time required for passing from one volume to a subsequent volume is generally of the order of 1-2 s.

As evidenced in FIGS. 2A and 2B, an accurate determination of the initial energy, Ek, of a hadron beam may be important because, if the position of the Bragg peak does not correspond to the actual position of the target tissue 40, substantial doses of hadrons may be delivered to healthy, sometimes vital, organs and may possibly endanger the health of a patient. The position of the Bragg peak may depend on the initial energy, Ek, of the hadron beam and/or on the nature and thicknesses of the traversed tissues. Besides determining the position of the target tissue within a patient, the computation of the initial energy, Ek, of a hadron beam yielding a position of the Bragg peak corresponding to the precise position of the target tissue may also require the preliminary characterization of the tissues traversed until reaching the target tissue 40. This characterization may be performed during a treatment plan established before (e.g., generally several days before) the actual hadron treatment. The actual hadron treatment may be divided in several sessions distributed over several weeks. A typical treatment plan may start by the acquisition of data, e.g., generally in the form of images of the subject of interest with a CT scan. The images thus acquired by a CT scan may be characterized, for example, by:
identifying the nature of the tissues represented on the images as a function of the X-rays absorption power of the tissues, e.g., based on the comparison of shades of grey of each tissue with a known grey scale; for example, a tissue may be one of fat, bone, muscle, water, air, or the like;
measuring the positions and thicknesses of each tissue along one or more hadron beam paths, Xp, from the skin to the target tissue;
based on their respective nature, attributing to each identified tissue a corresponding hadron stopping power ratio (HSPR);
calculating a tissue water equivalent path length, WEPLm, of each tissue m, with m=40 to 44 in the illustrated examples of FIGS. 2A and 2B, upstream of and including the target tissue, based on their respective HSPR and thicknesses;
adding the determined WEPLm of all tissues m to yield a WEPL40s of a target spot 40s located in the target tissue 40, said WEPL40s corresponding to the distance travelled by hadron beam from the skin to the target spot 40s; and
based on the WEPL40s, calculating the initial energy Ek of a hadron beam required for positioning the Bragg peak of the hadron beam at the target spot 40s.
   Said process steps may be repeated for several target spots defining the target tissue.

### Magnetic Resonance Imaging Device

A magnetic resonance imaging device 2 (MRI) generally implements a medical imaging technique based on the interactions of excitable atoms present in an organic tissue of a subject of interest with electromagnetic fields. When placed in a strong main magnetic field, B0, the spins of the nuclei of said excitable atoms typically precess around an axis aligned with the main magnetic field, B0, resulting in a net polarization at rest that is parallel to the main magnetic field, B0. The application of a pulse of radio frequency (RF) exciting magnetic field, B1, at the frequency of resonance, fL, called the Larmor frequency, of the excitable atoms in said main magnetic field, B0, may excite said atoms by tipping the net polarization vector sideways (e.g., with a so-called 90° pulse, B1-90) or to angles greater than 90° and even reverse it at 180° (e.g., with a so-called 180° pulse, B1-180). When the RF electromagnetic pulse is turned off, the spins of the nuclei of the excitable atoms generally return progressively to an equilibrium state yielding the net polarization at rest. During relaxation, the transverse vector component of the spins typically produces an oscillating magnetic field inducing a signal, which may be collected by antennas 2a located in close proximity to the anatomy under examination.

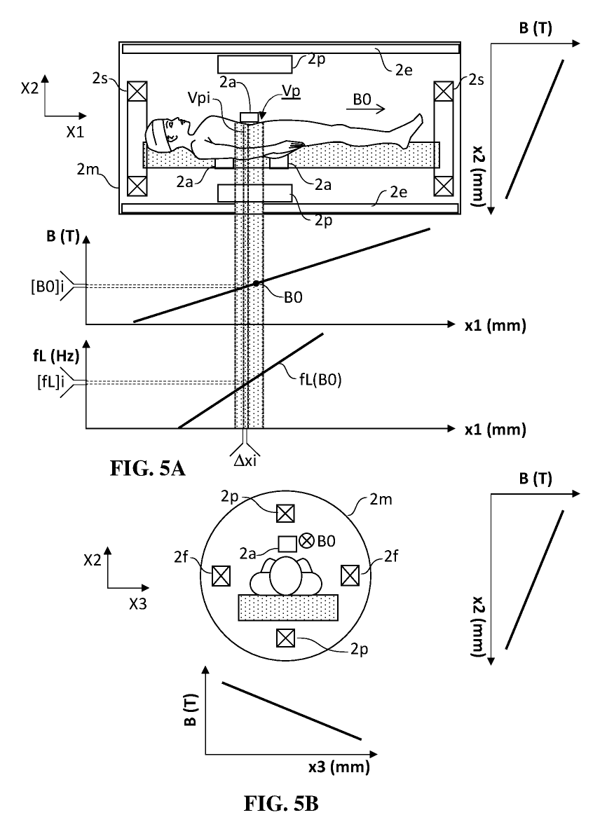

As shown in FIGS. 5A, 5B, 6A, and 6B, an MRI 2 usually comprises a main magnet unit 2m for creating a uniform main magnetic field, B0; radiofrequency (RF) excitation coils 2e for creating the RF-exciting magnetic field, B1; X1-, X2-, and X3-gradient coils, 2s, 2p, 2f, for creating magnetic gradients along the first, second, and third directions X1, X2, and X3, respectively; and antennas 2a, for receiving RF-signals emitted by excited atoms as they relax from their excited state back to their rest state. The main magnet may produce the main magnetic field, B0, and may be a permanent magnet or an electro-magnet (e.g., a supra-conductive magnet or not). An example of a suitable MRI includes, but is not limited to, a device described in U.S. Pat. No. 4,694,836, the entire disclosure of which is incorporated herein by reference as representative of an MRI used in the present disclosure.

As illustrated in FIG. 5A, an imaging slice or layer, Vpi, of thickness, Δxi, normal to the first direction, X1, can be selected by creating a magnetic field gradient along the first direction, X1. In FIG. 5A, the first direction, X1, is parallel to the axis Z defined by the lying position of the patient, yielding slices normal to said axis Z. In some embodiments, the first direction, X1, may be any direction, e.g., transverse to the axis Z, with slices extending at an angle with respect to the patient. As further shown in FIG. 5A, because the Larmor frequency, fL, of an excitable atom generally depends on the magnitude of the magnetic field it is exposed to, sending pulses of RF exciting magnetic field, B1, at a frequency range, [fL]i, may excite exclusively the excitable atoms which are exposed to a magnetic field range, [B0]i, which may be located in a slice or layer, Vpi, of thickness, Δxi. By varying the frequency bandwidth, [fL]i, of the pulses of RF exciting magnetic field, B1, the width, Δxi, and position of an imaging layer, Vpi, may be controlled. By repeating this operation on successive imaging layers, Vpi, an imaging volume, Vp, may be characterized and imaged.

To localize the spatial origin of the signals received by the antennas on a plane normal to the first direction, X1, magnetic gradients may be created successively along second and third directions, X2, X3, wherein X1 ⊥ X2 ⊥ X3, by activating the X2-, and X3-gradient coils 2p, 2f, as illustrated in FIG. 5B. Said gradients may provoke a phase gradient, Δφ, and a frequency gradient, Δf, in the spins of the excited nuclei as they relax, which may allow spatial encoding of the received signals in the second and third directions, X2, X3. A two-dimensional matrix may thus be acquired, producing k-space data, and an MR image may be created by performing a two-dimensional inverse Fourier transform. Other modes of acquiring and creating an MR image may be utilized concurrently with or alternatively to the mode described above.

The main magnetic field, B0, may be between 0.2 T and 7 T, e.g., between 1 T and 4 T. The radiofrequency (RF) excitation coils 2e may generate a magnetic field at a frequency range, [fL]i, around the Larmor frequencies, fL, of the atoms comprised within a slice of thickness, Δxi, and exposed to a main magnetic field range [B0i]. For atoms of hydrogen, the Larmor frequency per magnetic strength unit is approximately fL/B=42.6 MHz T⁻¹. For example, for hydrogen atoms exposed to a main magnetic field, B0=2 T, the Larmor frequency is approximately fL=85.2 MHz.

The MRI may be any of a closed-bore, open-bore, or wide-bore MRI type. A typical closed-bore MRI has a magnetic strength of 1.0 T through 3.0 T with a bore diameter of the order of 60 cm. An open-bore MRI, as illustrated in FIGS. 6A and 6B, has typically two main magnet poles 2m separated by a gap for accommodating a patient in a lying position, sitting position, or any other position suitable for imaging an imaging volume, Vp. The magnetic field of an open-bore MRI is usually between 0.2 T and 1.0 T. A wide-bore MRI is a kind of closed-bore MRI having a larger diameter.

### Hadron Therapy Device+MRI

As discussed previously with reference to FIG. 2B, the position and morphology of a target tissue 40 may evolve between a time, t0, of establishment of a treatment plan and a time, t1=t0+Δt3, of a treatment session, which may be separated by several days or weeks. A target spot 40si,j identified in the treatment plan as belonging to the target tissue 40p may not belong to the target tissue 40 anymore at the time, t0+Δt3, of the treatment session. The irradiation of said target spot may harm healthy tissues 43 instead of target tissues 40.

To avoid such incidents, a hadron therapy device (PT) 1 may be coupled to an imaging device, such as a magnetic resonance imaging device (MRI) 2. Such coupling may raise a number of challenges to overcome. For example, the correction of a hadron beam path, Xp, within a strong magnetic field, B0, of the MRI is a well-researched problem with proposed solutions.

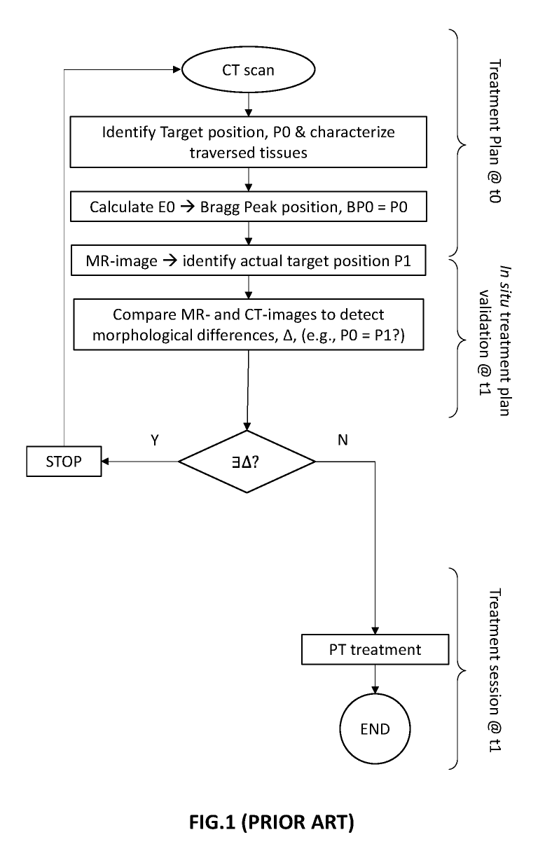

A PT-MRI apparatus may allow the morphologies and positions of the target tissue and surrounding tissues to be visualized, for example, on the day, t0+Δt3, of the treatment session for comparison with the corresponding morphologies and positions acquired during the establishment of a treatment plan at time, t0. As illustrated in the flowchart of FIG. 1, in cases having a discrepancy of the tissues morphologies and positions between the establishment of the treatment plan at time, t0, and the treatment session at time, t0+Δt3, the treatment session may be interrupted and a new treatment plan may be established with the definition of new target spots corresponding to the actual target tissue 40 to be irradiated by hadron beams of corrected energies and directions (in the example of FIG. 1, this procedure is represented by diamond box “∃Δ?”→Y→“STOP”). This represents a major improvement over carrying out a hadron therapy session based solely on information collected during the establishment of the treatment plan at time, t0, which may be obsolete at the time, t0+Δt3, of the treatment session.

Embodiments of the present disclosure may further improve the efficacy of a PT-MRI apparatus by providing the information required for correcting in situ the initial energies, Ek, and beam path, Xp, directions of the hadron beams, in case a change of morphology or position of the target tissue were detected. This may allow the treatment session to take place in spite of any changes detected in the target tissue 40.

The MRI used in embodiments of the present disclosure may be any of a closed-bore, open-bore, or wide-bore MRI type described above. An open MRI may provide open space in the gap separating the two main magnet poles 2m for orienting a hadron beam in almost any direction. Alternatively, openings or windows 2w transparent to hadrons may be provided on the main magnet units, as illustrated in the example of FIG. 6A. This configuration may allow the hadron beam to be parallel to B0. In another embodiment, a hadron beam may be oriented through the cavity of the tunnel formed by a closed bore MRI, or an annular window transparent to hadrons may extend parallel to a gantry substantially normal to the axis Z, over a wall of said tunnel, such that hadron beams may reach a target tissue with different angles. In embodiments where a fixed nozzle is used, the size of such opening or window may be reduced accordingly.

### MRI Imaging of Tissues and of Hadron Beam

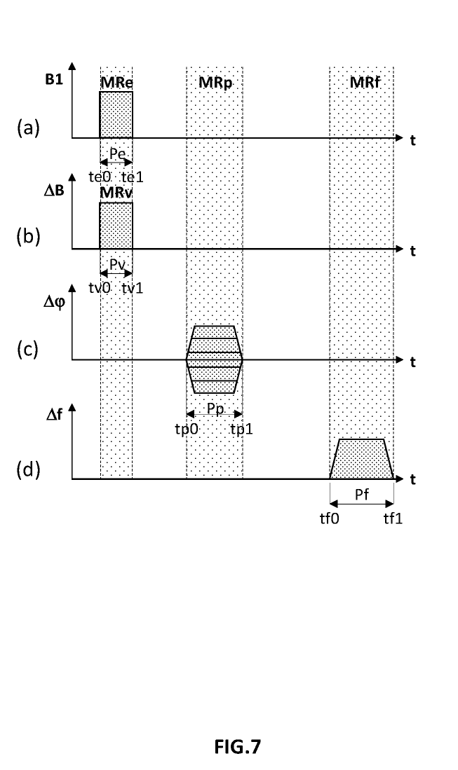

Acquisition of magnetic resonance data by a MRI for imaging a volume, Vp, may comprise the following steps illustrated in FIG. 7.
(A) an excitation step (MRe) illustrated in FIG. 7, step (a), for exciting the spin of the nuclei of excitable atoms A0, generally hydrogen; as shown in the example of FIG. 7, step (a), the excitation step (MRe) may be applied during a period, Pe=te1−te0;
(B) a layer selection step (MRv) illustrated in FIG. 7, step (b), for selecting an imaging layer, Vpi, of the imaging volume, Vp, of thickness, Δxi, measured along the first direction, X1; as shown in the example of FIG. 7, step (b), the layer selection step (MRv) may be applied during a period, Pv=tv1−tv0, wherein the periods Pe and Pv may be substantially simultaneous and equal;
(C) a phase gradient step (MRp) illustrated in FIG. 7, step (c), for localising along the second direction, X2, the origin of RF signals received by the antennas during relaxation of the excited spins; as shown in FIG. 7(c), the phase gradient step (MRp) is applied during a period, Pp=tp1−tp0, wherein generally, tp0≥te1; and
(D) a frequency gradient step (MRf) illustrated in FIG. 7, step (d), for localising along the third direction, X3, the origin of RF signals received by the antennas during relaxation of the excited spins; as shown in the example of FIG. 7, step (d), the frequency gradient step (MRf) may be applied during a period, Pf=tf1−tf0, wherein, e.g., tf0≥tp1.

The excitation step may comprise creating pulses of an excitation electromagnetic field, B1, with the RF unit 2e oscillating at a RF-frequency range, [fL]i, during an excitation period, Pe. The excitable atoms A0 present in an imaging layer, Vpi, of thickness, Δxi, are typically excited at their Larmor frequency, which may depend on the strength of the magnetic field to which are exposed, corresponding to a magnetic field range, [B0]i=[Bi,0, Bi,1] controlled by the magnetic gradient created along the first direction, X1 (and/or other directions) (as depicted in the example of FIG. 5A). The thickness, Δxi, may be varied as a function of the slope of the magnetic gradient and, in particular, by varying the band width of the RF-frequency range, [fL]i, applied by the RF unit 2e. Depending on the sequence and intensity of the RF-excitation electromagnetic field, B1, different types of excitations can be imposed onto the excitable atoms. The imaging volume, Vp, may generally be divided into several imaging layers, Vpi, sizes of which may be restricted along three dimensions by creating a magnetic gradient along, one, two, or three of the first, second, and third directions, X1, X2, X3. The thickness of the imaging volume may thus be controlled along said first, second, or third directions, X1, X2, X3, to define a slice (i.e., restricted over one direction only), an elongated prism (i.e., restricted over two directions), or a box (i.e., restricted over the three directions X1, X2, X3.

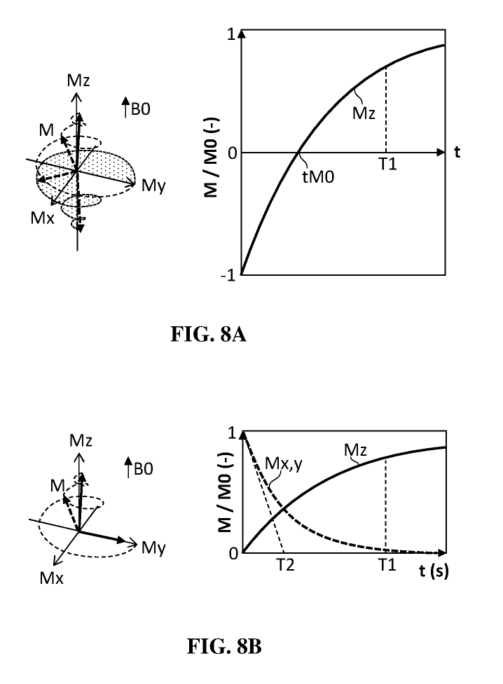

Absent an excitation electromagnetic field, B1, the net polarization vector of the excitable atoms A0 (e.g., hydrogen) of a tissue exposed to a main magnetic field, B0, parallel to the axis Z, is usually parallel to both B0 and Z, with a net polarization component, Mx,y, in the directions X and Y, which is generally zero as the spins precessing about the axis Z are out of phase and tend to compensate each other. upon excitation at their Larmor frequencies with an excitation electromagnetic field, B1, the precessing angle of the spins may increase, yielding a decrease in the Z-component, Mz, of the net polarization vector. Depending on the type of RF-excitation the spins can be brought in phase or not. In the former case, the net polarization component, Mx,y, increases as the spins of the excited atoms precess in phase. FIG. 8B illustrates an example of an excitable atom excited at 90°, yielding a zero Mz-component and maximum Mx,y-components, and then relaxing back to its rest state after the end of the excitation. The relaxation process and corresponding relaxation times, T1, T2 of this example are illustrated in the adjacent graph. FIG. 8A illustrates an example of an excitable atom excited at 180°. As the spin cannot be more excited than at 180°, this excited state is commonly referred to as a saturated state. However, a saturated state may be defined as an excitation state at an angle of at least 100° (and up to 180°). The corresponding relaxation process and relaxation times, T1, T2 of this example are illustrated in the adjacent graph of FIG. 8A.

Visualization by MRI of a hadron beam traversing tissues comprised within a MRI imaging volume, Vp, is not straightforward and has, as of the date of filing, generally has not been developed. Even proton beams, corresponding to hydrogen nuclei, cannot typically be visualized by MRI under normal conditions. Embodiments of the present disclosure may define specific conditions allowing a hadron beam and, in particular, the position of the Bragg peak of said hadron beam, to be identifiable on a MRI image of an imaging volume, Vp, traversed by said hadron beam.

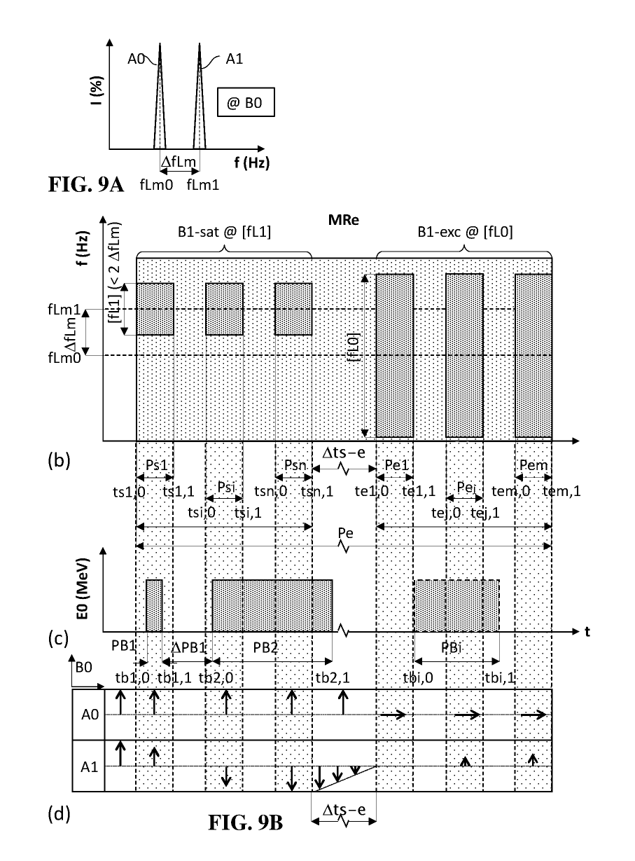

Some embodiments are based on the observation illustrated in the example of FIG. 9A, that the Larmor rest frequency, fLm0 of an excitable atom A0, such as hydrogen, in a tissue m exposed to a main magnetic field, B0, may be shifted to a value, fLm1, of a Larmor irradiated frequency, when said excitable atom interacted with a hadron beam passing in its direct vicinity (such atom is herein referred to as an irradiated excitable atom A1). The magnitude of the shift of the Larmor frequency may be expressed as an absolute value, ΔfLm=|fLm1−fLm0| expressed in Hz, or as a relative value ΔfLmr=ΔfLm/fLm0, expressed in ppm. Although the relative value, ΔfLmr, may be substantially independent of the magnetic field, B0, the magnitude of the shift, ΔfLm, may depend on the strength of the main magnetic field, B0, and/or on the nature of the tissue m comprising the excitable atoms and being traversed by a hadron beam. For example, the value of the shift, ΔfLm, of the Larmor frequency may be between 60 Hz and 6000 Hz, e.g., between 200 Hz and 1200 Hz in a main magnetic field, e.g., B0=1.5 T, corresponding, in this example, to a relative shift, ΔfLmr, of about 0.9 ppm to 93 ppm, e.g., about 3 ppm to 16 ppm.

The magnetic susceptibility of excitable atoms A0 may be modified by the effect of a hadron beam, yielding irradiated excitable atoms A1. The concentration of irradiated excitable atoms A1 may be a function of the energy deposited by said hadron beam in the tissues it traverses. As illustrated in the example of FIGS. 2A, 2B, and 2C, a hadron beam generally deposits almost all its energy at the level of the Bragg peak, which may be quite narrow. The magnetic susceptibility of the excitable atoms on and adjacent to the hadron beam path therefore may vary most at the level of the Bragg peak, resulting in a higher concentration of irradiated excitable atoms A1 at said level of the Bragg peak. Based on the treatment plan established earlier, the Bragg peak may be located within a target tissue 40 surrounding a target spot 40s. Embodiments of the present disclosure may use the foregoing mechanism for visualizing in an MR image of an imaging volume, Vp, of tissues at least the portion of the hadron beam at the level of the Bragg peak in the target tissue 40 and, in certain aspects, the whole of the hadron beam path from the outer surface, e.g., the skin of a patient, to the target tissue.

Some embodiments of the present disclosure may use the specific sequence applied in the excitation step, MRe, for the MR data acquisition as a function of the shift, ΔfLm, of the excitable atoms in a specific target tissue 40 and a specific synchronization of the hadron beam with the excitation step. The shift, ΔfLm, may be measured by nuclear resonance spectroscopy (MNR), the peaks corresponding to the excitation of excitable atoms A0 of the target tissue at rest and of irradiated excitable atoms A1 exposed to a hadron beam, yielding a spectrum as schematically illustrated in the example of FIG. 9A. Upon increasing the number of such RMN measurements, databases may soon be available with good approximations of the values of, ΔfLm, as a function of the tissues and of the main magnetic field, B0.

The excitation step, MRe, may comprise two main steps as illustrated in the example of FIG. 9B. First, a A1-saturation step may be performed on the irradiated excitable atom A1. The A1-saturation step may comprise emitting n bursts of a saturating electromagnetic field, B1-sat, which may oscillate at a frequency range, [fL1], of band width, b1<2·ΔfL40, and centred on the Larmor irradiated frequency, fL40,1. The band width b1 may exclude the Larmor rest frequency, fL40,0. The number n of bursts may be an integer greater than 0. The boundary of the frequency range, [fl1], closest to the Larmor frequency fLm0 of the excitable atoms A0 may be separated from the latter by a value of preferably at least ½ ΔfLm. The expression “centred on the [ . . . ] frequency fL40,1” does not restrict the position of fl40,1 to exactly the mid-point of the frequency range [fL1], but indicates that the Larmor frequency fL40,1 is comprised within a mid-portion of the range [fL1], separate from the upper and lower boundaries of said range. For example the Larmor frequency fL40,1 may be separated from the upper and lower boundaries of [fL1] by at least 20% of the range [fL1]. The period, Psi=tsi1−tsi0, of the bursts of B1-sat may be between 1 md and 20 ms, e.g., between 5 md and 15 ms. The bursts may be repeated at intervals, (ts(i+1)0−tsi1), e.g., between 1 and 50 ms, or between 5 and 20 ms.

The saturating electromagnetic field, B1-sat, may bring the nuclei of the irradiated excitable atoms (A1) to a saturated state, wherein a net polarization vector of the spins may be reversed at an angle between 100° and 180° with respect to the net polarization vector of the spins of said nuclei at rest (i.e., absent B1-sat). As illustrated in the example of FIG. 8A, a reversed angle of 180° may enhance the visibility of the hadron beam path, but lower angles may reduce data acquisition times. In one embodiment, the spins of the saturated atoms may not be brought into phase by the n bursts of B1-sat, such that the X- and Y-components, Mx,y, of the net polarization vector in the X- and Y-directions may remain substantially zero in the saturated state; this configuration is represented in the graph of the example of FIG. 8A.

At the end of the A1-saturation step, an A0-excitation step may be performed for exciting the excitable atoms A0, which are not substantially affected by the passage of the hadron beam. The A0-excitation step may comprise creating p bursts of an exciting electromagnetic field, B1-exc, oscillating at a frequency range, [fL0], and centred on the Larmor rest frequency, fL40,0. The same meaning of the term “centred” as defined above with respect to [fL1] applies mutatis mutandis to [fL0]. The number m of excitation bursts may be an integer greater than 0. The excitation step may bring to an excited state the excitable atoms (A0) which are not affected substantially by the hadron beam and which may therefore not have been brought to a saturated state by the saturating electromagnetic field, B1-sat. In some embodiments, the A0-excitation step may rotate the net polarization vector by about 90°, as illustrated in the graph of the example of FIG. 8B. During the A0-excitation step, the spins of the excitable atoms A0 may be brought into phase. Magnetic resonance data may be collected and images generated as explained above based on the RF-signals emitted by the excitable atoms A0 upon relaxation, based on either or both T1 and T2 relaxation times.

The period of time, Δts-e, separating the A1-saturation step from the A0-excitation step (i.e., separating the n^{th} of the B1-sat bursts, from the first of the p B1-exc bursts) may be important for the visualization of the hadron beam path. In one embodiment, the period of time, Δts-e, may be very short, e.g., as short as zero, such that when the excitation step starts, the irradiated excitable atoms A1 which are in or close to a saturated state may not react to the p bursts of B1-exc. In this embodiment, the period of time, Δts-e, may be not longer than half the longitudinal relaxation time, T1, of the excitable atoms A0, e.g., not longer than T1/3 or not longer than T1/4, which may be short enough for the irradiated excitable atoms A1 to be close enough to a saturated state to not respond substantially to the A0-excitation step. In one example, the period of time, Δts-e, may be not more than 100 ms/T, e.g., not more than 70 ms/T or not more than 50 ms/T.

In an alternative embodiment, wherein the A1-saturation step includes no phasing of the spins, resulting in a X- and Y-components, Mx,y, of the net polarization substantially equal to zero, the period of time, Δts-e, may be within ±20% of the time, tM0, at which the Z-component, Mz, of the net polarization vector, M, of the irradiated excitable atoms A1, is (approximately) zero, so that Mz may be too small for contributing to the RF-signals collected by the antennas. For example, the period of time, Δts-e, may be between 0.8 tM0 and 1.05 tM0. In one example, the period of time, Δts-e, may be not more than 50 ms/T. Using T2 weighed imaging therefore may not detect the relaxations of the irradiated excitable atoms A1.

A hadron therapy device may be provided suitable for directing a hadron beam along a beam path intersecting said target body in a number, N, of hadron pulses of pulse periods, Pbi, wherein, N may be an integer greater than 0. The hadron beam may have an initial energy, E0, e.g., previously determined during the establishment of a treatment plan for reaching the target tissue 40 at the level of an iso-energy layer, Vti, comprising target spots 40si,j (as depicted in the example of FIG. 4B). In order to visualize the hadron beam traversing the target tissue in a MRI image, the hadron beam pulses emitted by the hadron therapy device may be synchronized in a specific manner with the excitement step, MRe, of the MRI, as described above. A hyposignal representative of the hadron beam path may be visualized if a significant concentration of irradiated excitable atoms A1 is present during the A1-saturation step. This may be achieved, for example, if the N hadron pulses overlap with at least 50% of the n bursts of the saturating electromagnetic field, B1-sat during the A1-saturation step. This synchronization may account for the magnetic susceptibilities of the irradiated excitable atoms A1 returning rapidly to their original values after interruption of the hadron beam. For example, it is estimated that the irradiated excitable atoms A1 may return to their original state A0 on the order of us after interruption of the hadron beam.

As illustrated in FIG. 9B, step (c), the overlap may not be perfect. A hadron pulse PB1 may be shorter than an A1-saturation burst Ps1 and/or entirely or partly comprised in said burst. Several consecutive short hadron pulses may be comprised within an A1-saturation burst. Alternatively, a longer hadron pulse PB2 may overlap with several A1-saturation bursts Psi. Nevertheless, all the examples of FIG. 9B have an overlap of at least 50% (which may be at least 70%, at least 80%, at least 90%, or even 100%) between the N hadron pulses and the n A1-saturation bursts.

A hadron pulse does not generally consist of hadrons flowing continuously during the whole period PBi of the hadron pulse. A hadron pulse may instead be formed by consecutive trains of hadrons. In some embodiments, consecutive trains of hadrons separated from one another by a period of not more than 1.5 ms may form a single hadron pulse. Inversely, if two trains of hadrons are separated by a period of more than 1.5 ms, they may belong to two separate hadron pulses. For example, a synchro-cyclotron emitting a 10 μs-hadron train every 1 ms during 10 ms may form a single hadron pulse of period PBi=10 ms. Typically, a hadron pulse may have a period, PBi, e.g., between 10 μs and 30 ms, depending on the type of hadron source used. In one example, the hadron beam pulse period, PBi, may be between 1 ms and 10 ms. In another embodiment, the hadron beam pulse period, PBi, may be between 5 md and 20 ms. As discussed above with respect to the example of FIG. 4C, two consecutive hadron pulses may be separated from one another by a period, ΔPBi, for example, between 1 md and 20 ms, e.g., between 2 md and 10 ms.

FIG. 9B, step (d), illustrates schematically the spins of the excitable atoms A0 and A1 as the example excitation sequence proceeds. During the A1-saturation step carried out at a frequency range, [fL1], excluding the Larmor frequency, fLm0, of the excitable atoms A0, unaffected (or little affected) by the hadron beam, the net polarization vectors of the excitable atoms A0 may remain unaffected and is parallel to B0. The irradiated excitable atoms A1, on the other hand, may be excited to saturation, in that the Z-component, Mz, of their magnetic moment may be rotated by an angle between 100° and 180°, e.g., between 160° and 180°.

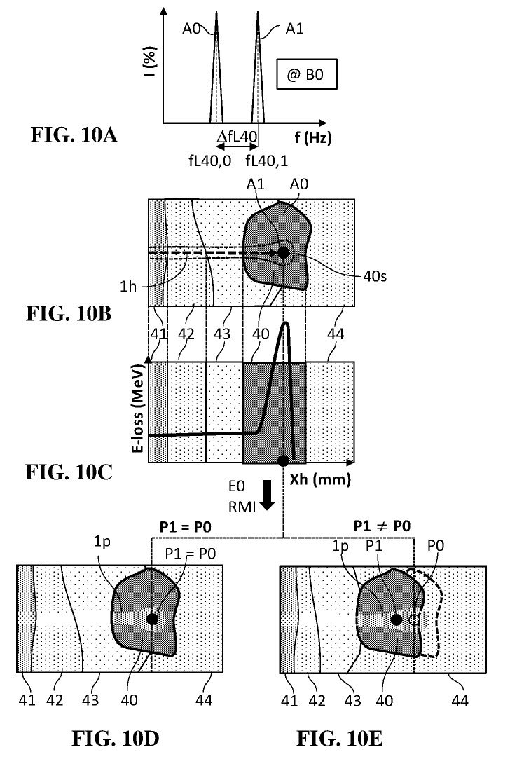

A time, Δts-e, after the end of the saturation step, the excitation step may be started. In FIG. 9B, step (d), the time, Δts-e, may correspond to about the time, tM0, required for the Z-component, Mz, of the net polarization vector of the irradiated excitable atoms A1 to become (approximately) zero. As discussed above, a time, Δts-e, e.g., not longer than T1/2, wherein T1 is the longitudinal relaxation time of the excitable atoms, A0, may alternatively be selected (as depicted in the example of FIG. 8A). The frequency range, [fL0], may or may not include the Larmor frequency, fLm, 1, of the irradiated excitable atoms A1, and the bandwidth of [fLm0], may be freely selected based on other requirements, such as the desired thickness, Δxi, of the imaging layer, Vpi. The spins of the excitable atoms A0 may be excited and rotated by an angle of, e.g., approximately 90° (as depicted in the examples of FIG. 8B and 9B, step (d)).The irradiated excitable atoms having a Mz value of about zero may not be excited by the magnetic field B1-exc and thus may emit substantially no RF signal receivable by the antennas 2a. In embodiments where the time, Δts-e, is shorter, the spins of the irradiated excitable atoms A1 may still be saturated and may not react to the excitation step B1-exc. The MR data thus acquired may yield an image wherein the zones comprising irradiated excitable atoms A1 may be visible as a hyposignal compared with the zones comprising non-irradiated excitable atoms A0, as discussed below with reference to FIGS. 10D and 10E. In order to capture the whole of a hadron beam path, the first direction, X1, defining the thickness, Δx1, of an imaging layer, Vpi, may be normal to the hadron beam 1h as shown e.g., in FIGS. 6A, 6B, and 10B, such that the hadron beam may be comprised in a single imaging layer.

FIG. 10A illustrates an example shift, ΔfL40, between the Larmor frequencies of excitable atoms A0 and irradiated excitable atoms A1 located in a target tissue 40 exposed to a main magnetic field, B0. The target tissue 40 may be a tumour composed of cancerous cells. FIG. 10B illustrates schematically an example image of the tissues traversed by a hadron beam 1h represented by a thick dashed line reaching a target spot 40s located in the target tissue 40. The hadron beam 1h may cross a number of healthy tissues 41-43 before reaching the target tissue 40 and the target spot 40s. The tissue 41 may, for example, be the skin of a patient. The thin dotted line represents an irradiated volume surrounding the hadron beam 1h containing the irradiated atoms A1 whose magnetic susceptibility may be modified by the passage of the hadron beam and that may be characterized by a Larmor frequency, fLm1, with m=40 to 43. Outside said irradiated volume, the magnetic susceptibility of the excitable atoms A0 may not be affected significantly by the hadron beam, and their Larmor frequency is fLm0, with m=40-43. Tissue 44 is a healthy tissue, e.g., a vital tissue, located downstream of the target tissue 40, and may not be reached by the hadron beam.

FIG. 10C shows an example energy loss curve of the hadron beam 1h as it travels across the tissues until reaching the target spot in the target tissue. The hadron beam has an initial energy, E0, (i.e., before reaching the first tissue 41 along its beam path) which may have been determined previously during the establishment of a treatment plan. If the treatment plan was performed accurately, and if the relative positions and morphologies of the tissues 40-43 traversed by the hadron beam have not changed since the establishment of the treatment plan, the Bragg peak of a hadron beam of initial energy, E0, may fall at the position of the target spot 40s. An example of this situation is illustrated in FIG. 10D.

As discussed above, however, it is possible that the sizes and positions of the tissues traversed by a hadron beam may change between the day, t0, a treatment plan had been established and the day, t1, of a hadron therapy session. FIG. 10E illustrates an example where the tissues 42 and 43 located upstream from the target tissue 40 have shrunk between t0 and t1. Tissues 42 and 43 may, for example, be fat and/or muscles which can easily shrink during an illness. Consequently, the target tissue may have moved closer to the upstream boundary of the treated anatomy and the distance the hadron beam must travel across tissues until the actual position of the target spot 40s(t1) may have decreased accordingly. Irradiation of the tissues with a hadron beam of initial energy, E0, may reach beyond the actual position of the target spot. As illustrated in FIG. 1, the identification of such mismatch between the planned position P0 and the actual position P1 in existing methods generally leads to the interruption of the treatment session and to the establishment of a new treatment plan, which may waste precious time and resources.

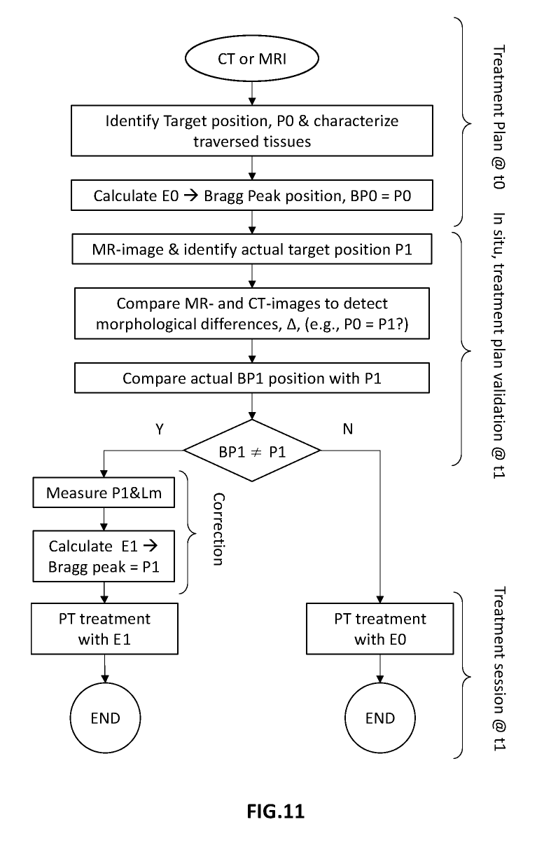

As discussed supra in relation with FIG. 1 (labeled “∃ Δ ?”→Y therein), absent the position of the Bragg peak of a hadron beam of initial energy, E0, on the day t1 of a hadron therapy session, if the MR images reveal any change of morphology or position of the tissues surrounding and including the target tissue since the day t0 of the treatment plan, the hadron therapy session generally must be stopped and a new treatment plan established. As illustrated in FIG. 11, by visualizing the hadron beam obtained by embodiments of the present disclosure, a mismatch may be identified between the position of the Bragg peak (BP) and the position, P1, of the target spot (even if P1=P0). Accordingly, embodiments of the present disclosure may allow for correcting in situ the initial energy, E1, such that the Bragg peak falls on the position P1 of the target spot. The correction involves the measurement of the position P1 of the target spot, and of the thicknesses, Lm, with m=40-43 as shown in the example of FIGS. 2A, 2B, and 2C, of the various tissues the hadron beam must traverse to reach the target spot 40s, to determine the distance the hadron beam must travel through the tissues to reach the target spot. Through the determination of the corresponding WEPLs as described above, one may calculate the initial energy, E1, such that the position of the Bragg peak of the hadron beam overlaps with the position, P1, of the target spot 40s. The treatment may thus proceed the same day with the corrected initial energy, E1. This may provide economical benefits as well as improve the health of the patients.

The doses deposited onto the tissues for visualizing the hadron beam path must generally be low, because, in case of a change of morphology of the tissues, a full therapeutic dose reaching healthy tissues may be extremely detrimental to the health of a patient. Accordingly, the hadron doses deposited for the visualization of the hadron beam may be substantially lower than the therapeutic doses required for treating the target tissue and may have substantially no therapeutic effects. As discussed with respect to FIG. 4C, this may be achieved either by irradiating few target spots, e.g., irradiating 1% to 40% of the target spots of an iso-energy layer, Vti, e.g., 5% to 30% or 10% to 20%. Alternatively or concomitantly, target spots may be irradiated with a hadron beam having an intensity substantially lower than prescribed by the treatment plan. Finally, the irradiation time, ti, may also be reduced, e.g., to the minimum required for acquiring a MR image. In these conditions, the validation of the treatment plan is generally safe for the patient, even if a correction of the initial energy is then required. For example, some embodiments may irradiate only a selection of the target spots 40si,j of the target tissue to yield the relative positions of the Bragg peak, BP1, and the corresponding target spot, 40s, to calculate the initial energy, E1, which may be used during the treatment session to treat all the target spots 40si,j of an iso-energy volume, Vti. The initial energies required for treating target spots, 40(i+1),j, etc., in subsequent iso-energy volumes, Vt(i+1), etc., may either be extrapolated from the initial energy, E1, and/or determined for the iso-energy volume, Vti, or, alternatively or additionally, a selection of target spots 40(i+1),j, etc., of the subsequent energy volumes, Vt(i+1), etc., may be tested as described above.

Embodiments of the present disclosure also include a medical apparatus for carrying out the foregoing method of visualizing a hadron beam together with the target tissue it must irradiate. The medical apparatus may comprise:
(a) a hadron source adapted for irradiating a target tissue 40 with a hadron beam 1h having a beam energy, Ek, with k=0 or 1, along a beam path in a number, N, of hadron pulses;
(b) a magnetic resonance imaging device (MRI) for the acquisition of magnetic resonance (MR) images within an imaging volume, Vp, including the target tissue 40, as the target tissue is being irradiated,
(c) a controller configured for controlling the hadron source and for acquiring magnetic resonance images by implementing the following steps:
   creating a main magnetic field, B0, in the imaging volume, Vp, including the target tissue 40,
   creating n bursts of a saturating electromagnetic field, B1-sat, wherein n is an integer greater than 0, which may oscillate at a frequency range, [fL1], of band width, b1<2·ΔfL40, and centred on the Larmor irradiated frequency, fL40,1, and excluding the Larmor rest frequency, fL40,0, wherein
      fL40,0 is the rest Larmor frequency of excitable atoms A0 present in the target tissue 40;
      fL40,1 is the irradiated Larmor frequency of irradiated excitable atoms A1 defined as the excitable atoms A0 present in the same target tissue, and exposed to the effects of a hadron beam of initial energy, E0, traversing said target tissue positioned in the same magnetic field, B0, and wherein
      ΔfL40=|fL40,1−fL40,0|;
   after a time, Δts-e, following the nth burst B1-sat, creating p bursts of an exciting electromagnetic field, B1-exc, oscillating at a frequency range, [fL0], centred on the Larmor rest frequency, fL40,0, wherein m is an integer greater than 0;
   directing a hadron beam having the initial energy, E0, along a beam path intersecting said target tissue in a number, N, of hadron pulses of pulse periods, Pbi , wherein, N is an integer greater than 0;
(d) a display for representing the target tissue from the magnetic resonance data acquired by the MRI within the imaging volume, Vp, and for visualizing the beam path in the target tissue as a hyposignal 1p, weaker than the signal generated by the excitable atoms A0 which may not be exposed significantly to the effects of the hadron beam,
   characterized in that, said controller may be further configured for synchronizing the N hadron pulses to overlap with at least 50% of the n bursts of the saturating electromagnetic field, B1-sat.

1.-10. (canceled)

11. A computer-implemented method for displaying, on a computer display, a hadron beam traversing an organic body, wherein the hadron beam is provided by a hadron source configured to direct the hadron beam with an initial energy along a beam path intersecting a target tissue in an imaging volume, the method comprising:
determining a Larmor rest frequency of first excitable atoms in the target tissue of the organic body, wherein the Larmor rest frequency represents a frequency of the first excitable atoms in a uniform magnetic field;
determining a Larmor irradiated frequency of the first excitable atoms, wherein the Larmor irradiated frequency represents a frequency of the first excitable atoms in the hadron beam with the initial energy;
calculating a frequency shift in the target tissue based on the Larmor rest frequency and the Larmor irradiated frequency;
acquiring, using a magnetic resonance imaging device, magnetic resonance data associated with the imaging volume that includes the target tissue and that is positioned in the uniform magnetic field, wherein acquiring the magnetic resonance data further includes:
generating one or more bursts of a saturating
electromagnetic field oscillating at a first frequency range having a bandwidth, centred on the Larmor irradiated frequency, and excluding the Larmor rest frequency, such that nuclei of the first excitable atoms move to a saturated state with a first net polarization vector of spins reversed at an angle between 100° and 180° with respect to a second net polarization vector of the spins at rest, and
generating one or more bursts of an exciting electromagnetic field oscillating at a second frequency range centred on the Larmor rest frequency, such that nuclei of second excitable atoms not affected by the hadron beam and not in the saturated state move to an excited state;
directing the hadron beam with the initial energy along the beam path in one or more hadron pulses having one or more pulse periods, wherein one of the pulse periods overlaps with at least a percentage of the bursts of the saturating electromagnetic field;
representing, on the display, the organic body based on the magnetic resonance data;
displaying, on the display, the beam path as a hyposignal, the hyposignal being weaker than a signal generated by the second excitable atoms.

12. The method of claim 11, wherein the percentage is 50%.

13. The method of claim 11, wherein the percentage is 70%.

14. The method of claim 11, wherein the percentage is 90%.

15. The method of claim 11, wherein one of the pulse periods is in phase with the bursts of the saturating electromagnetic field.

16. The method of claim 11, wherein one or more of the pulse periods are between 10 μs and 30 ms.

17. The method of claim 16, wherein one or more of the pulse periods are between 5 ms and 20 ms.

18. The method of claim 11, wherein the one or more hadron pulses comprise at least two pulses, and wherein the two pulses are separated by a separation period.

19. The method of claim 18, wherein the separation period is between 1 ms and 20 ms.

20. The method of claim 11, wherein the bursts of the saturating electromagnetic field each have a period between 1 ms and 20 ms.

21. The method of claim 11, wherein the frequency shift is between 60 Hz and 6000 Hz.

22. The method of claim 21, wherein the frequency shift is between 200 Hz and 1200 Hz.

23. The method of claim 11, wherein the frequency shift is between 0.9 ppm and 93 ppm.

24. The method of claim 23, wherein the frequency shift is between 3 ppm and 16 ppm.

25. The method of claim 11, wherein a time period separation a last burst of the one or more bursts of the saturating electromagnetic field and a first burse of the one or more bursts of the exciting electromagnetic field is not more than 50% of a longitudinal relaxation time of the second excitable atoms.

26. The method of claim 11, wherein a time period separation a last burst of the one or more bursts of the saturating electromagnetic field and a first burse of the one or more bursts of the exciting electromagnetic field is within 20% of a time required for a longitudinal component of the first net polarization vector to move from the saturated state to zero.

27. The method of claim 11, wherein the one or more bursts of the saturating electromagnetic field are adiabatic bursts.

28. The method of claim 11, the imaging volume is controlled by generating a magnetic gradient along at least one of a first direction, a second direction normal to the first direction, and a third direction normal to the first direction and the second direction to control a thickness of the imaging volume along the first direction, the second direction, or the third direction.

29. The method of claim 11, further comprising:
establishing a treatment plan including the initial energy;
comparing, using the display, morphology and thicknesses of tissues traversed by the hadron beam;
displaying, on the display, the position of a Bragg peak of the hadron beam; and
when the position of the Bragg peak and a position of the target tissue differ by more than a threshold: correcting the initial energy such that the position of the Bragg peak and the position of the target tissue are within the second threshold

30. A medical apparatus comprising:
a hadron source for irradiating a target tissue with a hadron beam having an initial energy along a beam path in one or more hadron pulses;
a magnetic resonance imaging device for acquiring, during irradiation, magnetic resonance images within an imaging volume including the target tissue;
a controller configured to:
generate a main magnetic field in the imaging volume,
generate one or more bursts of a saturating electromagnetic field oscillating at a first frequency range having a bandwidth, centred on a Larmor irradiated frequency of first excitable atoms in the target tissue, and excluding a Larmor rest frequency of first excitable atoms in the target tissue,
after a last burst of the one or more bursts of a saturating electromagnetic field, generate one or more bursts of an exciting electromagnetic field oscillating at a second frequency range centred on the Larmor rest frequency, and
directing the hadron beam having the initial energy along the beam path intersecting the target tissue in one or more hadron pulses having one or more pulse periods, wherein one of the pulse periods overlaps with at least 50% of the bursts of the saturating electromagnetic field; and
a display for displaying the target tissue based on the magnetic resonance images and for visualizing the beam path in the target tissue as a hyposignal, the hyposignal being weaker than a signal generated by the second excitable atoms.
